(19) United States
(12) Patent Application Publication
(10) Pub. No.: US 2008/0140078 A1
(43) Pub. Date: Jun. 12, 2008

(54) SURGICAL TOOLS FOR USE IN DEPLOYING BONE REPAIR DEVICES

(75) Inventors: Charles L. Nelson, Santa Rosa, CA (US); Kai U. Mazur, Santa Rosa, CA (US); Heber Saravia, Santa Rosa, CA (US); Christopher Staudenmayer, Santa Rosa, CA (US)

Correspondence Address: SHAY GLENN LLP, 2755 CAMPUS DRIVE, SUITE 210, SAN MATEO, CA, 94403, US

(73) Assignee: SONOMA ORTHOPEDIC PRODUCTS, INC., SANTA ROSA, CA (US)

(21) Appl. No.: 11/944,379

(22) Filed: Nov. 21, 2007

Related U.S. Application Data
(60) Provisional application No. 60/866,976, filed on Nov. 22, 2006.

(51) Int. Cl. A61B 17/58

(52) U.S. Cl. 606/80; 606/79

(57) ABSTRACT

The invention is generally directed to tools, systems and methods of preparing a bone site prior to deployment of bone repair devices.

In one embodiment, a bone cutting instrument is provided with a generally rigid arcuate tube having a generally fixed radius and a lumen therethrough, a flexible drive shaft configured to be slidably and rotably received within the tube lumen, and a cutter head attached to an end of the drive shaft, whereby the shaft and cutter head may be first advanced together with the tube in an arcuate manner to cut an arcuate path in a bone, and then advanced in a telescoping manner relative to the tube being held in a generally fixed position to cut a straight path in the bone.

Methods of forming a passage in a bone include advancing a cutter head into a bone along a curved path having a generally constant radius, and continuing to advance the cutter along a generally straight path extending from the curved path.

### CROSS-REFERENCE

This application claims the benefit under 35 U.S.C. § 119 of the following U.S. provisional application, the disclosure of which is incorporated herein by reference: U.S. Ser. No. 60/866,976, “SURGICAL TOOLS FOR USE IN DEPLOYING BONE REPAIR DEVICES,” filed Nov. 22, 2006.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

1. Field of the Invention

The invention relates to tools used to access and prepare bone sites during reconstructive orthopedic surgery.

2. Description of the Prior Art

Currently available tools for arthroscopic surgery include, for example, that described in U.S. Pat. No. 4,007,528 to Shea et al. for High Speed Bone Drill. The Shea device is a high-speed, electric motor contained bone drill with an elongate drive tube into which most of the length of the burr or reamer shank engages. U.S. Pat. No. 5,913,867 to Dion for Surgical Instrument describes a surgical instrument having an inner tube which rotates within an outer tube and carries a surgical tool that includes a burr or reamer for cutting tissue exposed to the burr through an opening in the outer tube. U.S. Pat. No. 6,179,839 to Weiss et al. for Bone Fusion Apparatus and Method includes a description of a bone rasp, burr or reamer which can be used to rasp or burr bone in a precise location where bone fusion is to occur. U.S. Pat. No. 7,118,574 to Patel et al. for Arthroscopic Bone Burr Device describes an arthroscopic bone burr having an articulated sheath tube. The articulated sheath may be articulated angularly and transversely relative to the housing by a ball-type joint, a flange-in-socket with sufficient play to permit displacement of the sheath tube, or by use of an elastomer.

### SUMMARY OF THE INVENTION

The invention is generally directed to tools, systems and methods of preparing a bone site prior to deployment of bone repair devices, such as the devices described in the co-pending U.S. application Ser. No. 11/383,269, filed May 15, 2006, and 60/867,011 filed Nov. 22, 2006.

In some embodiments of the invention, a bone cutting instrument includes a generally rigid arcuate tube having a generally fixed radius and a lumen therethrough, a flexible drive shaft configured to be slidably- and rotably-received within the tube lumen, and a cutter head that is attached to an end of the drive shaft. The shaft and cutter head are configured such that they may be first advanced together with the tube in an arcuate manner to cut an arcuate path in a bone, and then advanced in a telescoping manner relative to the tube being held in a generally fixed position to cut a straight path in the bone.

Some embodiments of the bone-cutting instrument further include a jig that is configured to be coupled with the arcuate tube for alternately advancing the tube in an arcuate manner and for holding the tube in a generally fixed position. In some of these embodiments with a jig, the jig is configured to be handheld. In some embodiments with a jig, the jig is configured to be mounted in a fixed position relative to a surgical station.

With regard to the cutter, in some embodiments of the bone-cutting instrument, the cutter has a rounded end adjacent to the arcuate tube. In some embodiments, the cutter has a non-flat shape on an end adjacent to the arcuate tube, and in some embodiments the cutter has an approximately spherical shape.

In some embodiments of the instrument, the cutter head and drive shaft include a continuous lumen therethrough. And some of these particular embodiments further include a guide wire configured to be received through the lumen in the cutter head and drive shaft.

Embodiments of the invention also include a method of using the bone-cutting instrument summarized above to form a passage in a bone. The method includes advancing a cutter head into a bone along a curved path having a generally constant radius, and continuing to advance the cutter along a generally straight path extending from the curved path.

In some embodiments of the method of forming a passage in a bone, the generally straight path is generally along a portion of an intramedullary canal of the bone. In some embodiments of the method, the curved path extends from an opening in a fractured bone into the bone. In some these latter embodiments the opening is in a bony protuberance on an end of a radius bone.

Some embodiments of the method of forming a passage in a bone further include advancing a curved trocar into the bone prior to advancing the cutter head into the bone. In some of these particular embodiments, the method further includes advancing a guidewire along a path formed in the bone by the curved trocar and then using the guide wire to guide the cutter head along the curved path and the generally straight path.

Some embodiments of the method of forming a passage in a bone further include creating bone chips inside the bone as the cutter is advanced. These particular embodiments may further include removing the cutter head from the bone while leaving a majority of the bone chips in the paths formed in the bone.

In some embodiments of the method, advancing of the cutter head along the curved path includes pivoting the cutter head, a flexible drive shaft of the cutter head and a curved tube receiving the drive shaft together about a common pivot point.

In some embodiments of the method, advancing of the cutter head along the generally straight path includes holding a curved tube in a generally fixed position while extending a cutter head drive shaft from a lumen in the fixed tube.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an illustration of a device suitable for accessing an interior of a bone; the device is configured to have a reamer at one end;

FIG. 2 is a close-up illustration of a reamer head from the device depicted in FIG. 1;

FIGS. 3a-3b are cross-sections of the reamer-head and the reamer shaft;

FIG. 4 is a close-up of the reamer head;

FIG. 5 is a depiction of an individual reamer and a reamer placed in an arc cannula of a surgical station;

FIGS. 6a-6b are images of an arm with a reamer positioned for entry into target bone, and an arm with the reamer advancing into the bone space;

FIGS. 7a-7b are fluoroscans illustrating a reamer advancing into the bone space of a patient; guides are visible;

### DETAILED DESCRIPTION OF THE INVENTION

By way of background and to provide context for the invention, it may be useful to understand that bone is often described as a specialized connective tissue that serves three major functions anatomically. First, bone provides a mechanical function by providing structure and muscular attachment for movement. Second, bone provides a metabolic function by providing a reserve for calcium and phosphate. Finally, bone provides a protective function by enclosing bone marrow and vital organs. Bones can be categorized as long bones (e.g. radius, femur, tibia and humerus) and flat bones (e.g. skull, scapula and mandible). Each bone type has a different embryological template. Further each bone type contains cortical and trabecular bone in varying proportions.

Cortical bone (compact) forms the shaft, or diaphysis, of long bones and the outer shell of flat bones. The cortical bone provides the main mechanical and protective function. The trabecular bone (cancellous) is found at the end of the long bones, or the epiphysis, and inside the cortex of flat bones. The trabecular bone consists of a network of interconnecting trabecular plates and rods and is the major site of bone remodeling and resorption for mineral homeostasis. During development, the zone of growth between the epiphysis and diaphysis is the metaphysis. Finally, woven bone, which lacks the organized structure of cortical or cancellous bone, is the first bone laid down during fracture repair. Once a bone is fractured, the bone segments are positioned in proximity to each other in a manner that enables woven bone to be laid down on the surface of the fracture. This description of anatomy and physiology is provided in order to facilitate an understanding of the invention. Persons of skill in the art will appreciate that the scope and nature of the invention is not limited by the anatomy discussion provided. Further, it will be appreciated there can be variations in anatomical characteristics of an individual, as a result of a variety of factors, which are not described herein.

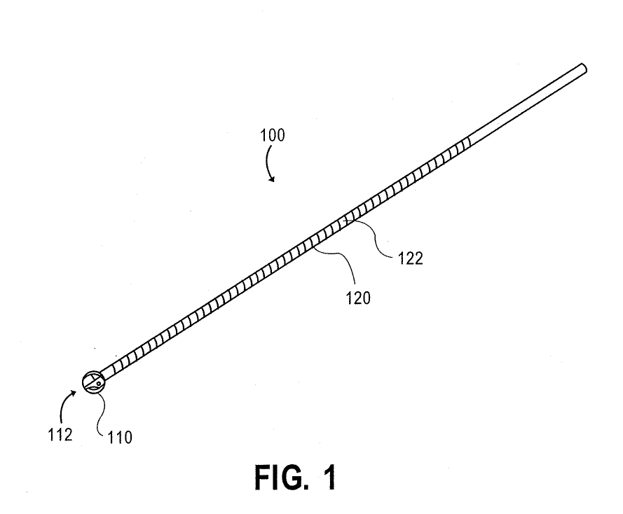

Turning now to FIG. 1, an illustration of a device 100 suitable for accessing an interior of a bone; the device 100 is configured to have a bone cutting element 110 at a distal end 112 which engages the tissue to be breached. Although element 110 will be referred to hereinafter as a reamer, it may also be called a burr, drill, rasp, grinder or by similar terminology, as it may be used to cut a new hole or enlarge an existing hole. The shaft 120 of the device 100 is configured to be flexible in bending, rigid in torsion and configure to telescope into a bone. The shaft 120 can be configured to have double counter opposed helical cuts, a welded spring design, nitinol tubes and/or elastic hardened steels such as piano wire. The device 100 is comprised of three main sections, a reamer, a flexible shaft, and a drill driver hub. The drill driver hub provides a rigid, crush-resistant attachment for a Jacob's or other form of a drill chuck. The shape of the drill hub may be hexagonal, circular, square, triangular, or any shape that involves a radius (e.g. elliptical) or a polygon.

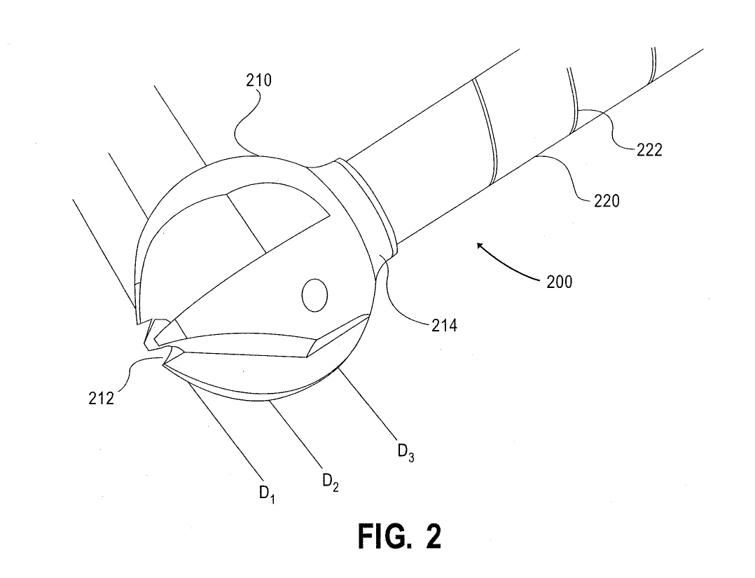

FIG. 2 is a close-up illustration of a reamer head 210 from the device 200 depicted in FIG. 1. In this embodiment, the reamer is shaped in the form of a pear. The front, distal-most to the user, section 212 has the narrowest diameter d1 and acts as a pilot as well as achieving initial bone removal for the larger diameter d3 of the reamer which occurs, in this depiction, just past the mid-point. The diameter of the reamer increases from d1 to d2 to d3. This configuration improves the cutting performance and quality of the resultant hole in the bone. Additionally, this configuration preserves the inherent viability of the bone tissue, and increases the longevity of the tool. The base of the reamer d3 is a larger diameter and has a larger, substantially spherical, cross-section. The spherical cross-section of the base creates a continuous radius of circular cross-section from the access point of a patient through an exterior cortex of bone into an intramedullary space of bone. In some embodiments, the continuous diameter and circular cross-section is important so that the implant can be smoothly and easily placed within the bone. The reamer is adapted to connect integrally, or removably, with the shaft at its proximal end 214.

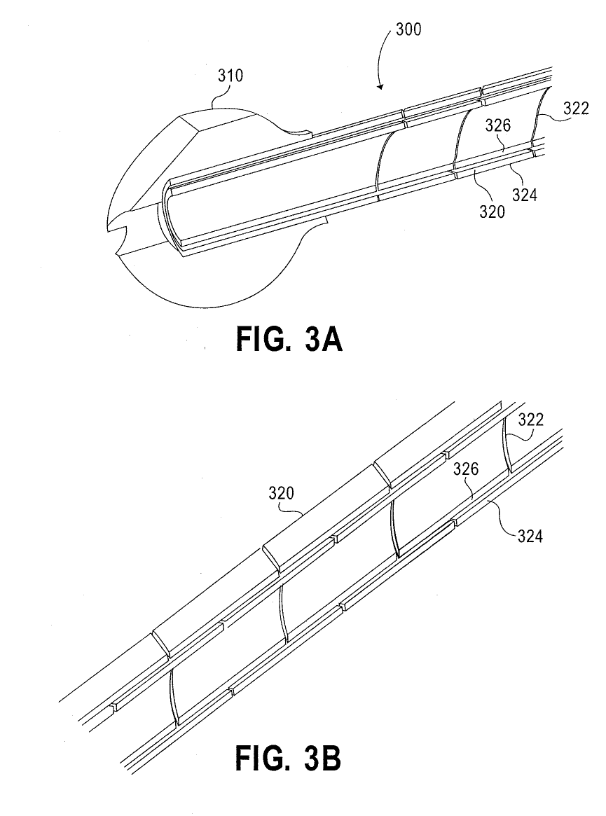

FIG. 3a is a cross-section of the reamer-head in an embodiment where the reamer is a separate piece from the shaft. The shaft 320 is also depicted in cross-section as having a first tubular section 324 surrounding an interior tubular section 326. The helical cuts 322 are configured as shown in FIG. 3a such that the cuts of both the inner and outer tubular members are lined-up at the same location. As shown in FIG. 3b, the helical cuts from the interior and exterior tubular section do not line up. In further embodiments (not shown), the reamer shaft may be formed by helically winding three coaxial layers together. The first and third layers (i.e. the inner and outmost layers) may be wound in a direction that provides the most torque transmission in the bone cutting direction of the reamer head, and the second layer (i.e. the middle layer) wound in the opposite direction to provide optimum torque transmission in the reverse rotational direction. The various helical layers provide a reamer shaft that is strong in torsion yet flexible enough to spin inside a curved canula and bend around a radius, as is described below.

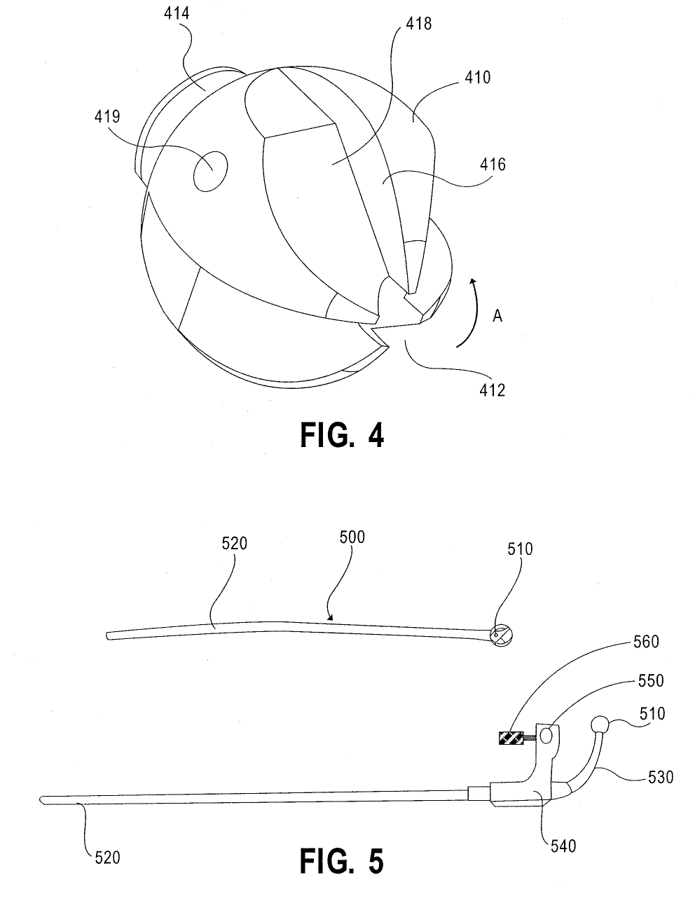

FIG. 4 is a close-up of the reamer head. As discussed above, in this embodiment the reamer head can be described as pear-shaped (as opposed to spherical) because it has a gradually changing diameter along its length, with the widest portion being approximately proximal the midline. The distal end of the reamer head (i.e., the part of the reamer that first makes contact with target tissue) does not evenly increase in diameter to the midline (like a sphere), rather the diameter begins small, the diameter change flattens out, and then increases again. Channels 416 are also provided to assist in cutting and clearing away tissue. Curved recessed faces 418 are also provided. For cutting, the reamer shown in FIG. 4 spins in the direction shown by Arrow A, and may be operated in the opposite direction when removing the reamer from the bone. Typical operating speeds of this reamer are in the range of 600 to 1200 RPM, driven for example by an electric, pneumatic or hydraulic drill motor. The reamer may also be operated by hand, typically at speed of less than 100 RPM. An aperture 412 is provided through the center of the reamer head to receive a guidewire. An additional aperture 419 is provided to receive a pin which engages the head to the flexible shaft to secure the head 110 to flexible shaft 520. In an alternative embodiment, the head 110 is laser-welded to the shaft 520.

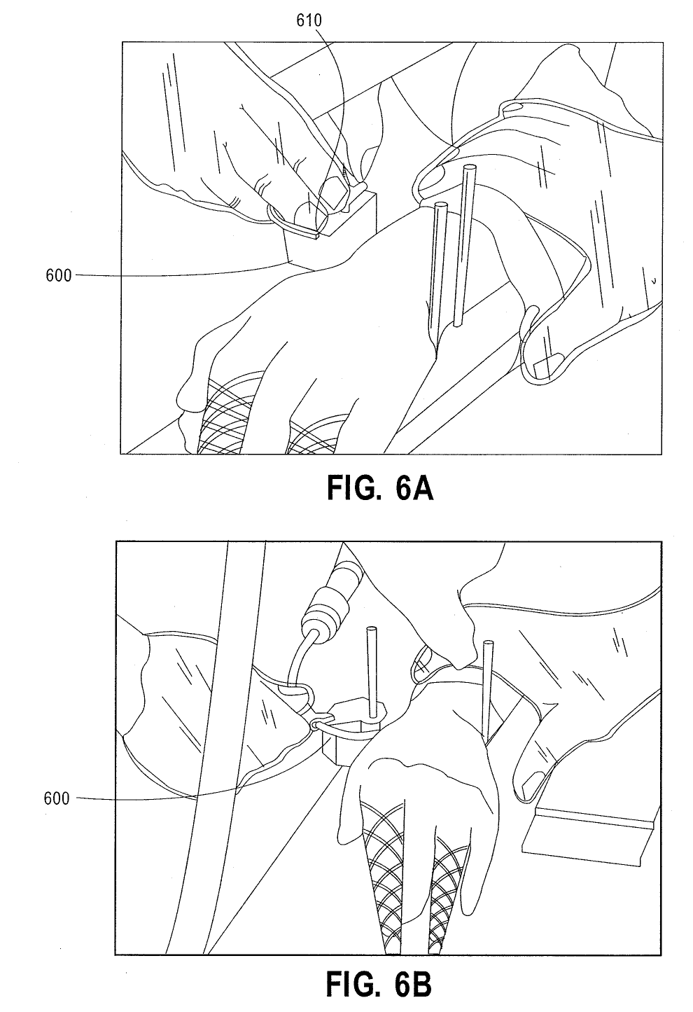

FIG. 5 is a depiction of an individual reamer 500, and also a reamer placed in an arc cannula 530 of a surgical station. In this embodiment, the arc canula 530 is a tube having a generally constant radius and a lumen therethrough for slidably and rotably receiving the drive shaft 520 of reamer 500. In this embodiment, the outer diameter of canula 530 is smaller than the diameter of the reamer head 510 so that the canula may follow reamer head 510 into the bone.

In operation, shaft 520 of reamer 500 is threaded through the lumen of arc canula 530. The end of shaft 520 opposite reamer head 510 is connected to the chuck of a drill motor for rotably driving the reamer 500. Arc canula 530 may be rigidly coupled to a mounting block 540 as shown. Mounting block 540 may include a pivot hole 550 located at the center of the radius of arc canula 530 for slidably and rotably receiving a fixed mounting post (not shown). With this arrangement, mounting block 540 (together with arc canula 530, reamer head 510 and reamer shaft 520) may be rotated about the fixed mounting post while reamer 500 is spinning to form a curved passage in the bone having the same radius of curvature as arc canula 530. To form a straight passage, thumbscrew 560 of mounting block 540 may be tightened against the post to hold mounting block 540 and arc canula 530 in a fixed position as reamer shaft 520 is advanced through canula 530. Alternative mounting arrangements may be employed, such as using a mounting block having a handle (not shown) so that it may be hand held rather than coupled to a mounting post. See copending application 60/866,920 to Jobson for SURGICAL STATION FOR ORTHOPEDIC RECONSTRUCTIVE SURGERY for additional details pertaining to the arc cannula.

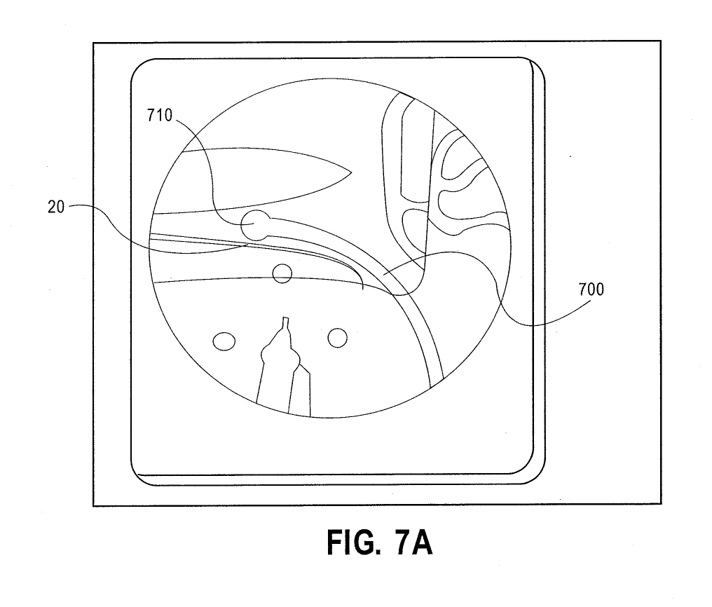

FIGS. 6a-6b are images of an arm of a patient 10 with a reamer 610 positioned for entry into target bone, and an arm of a patient with the reamer advancing into the bone space.

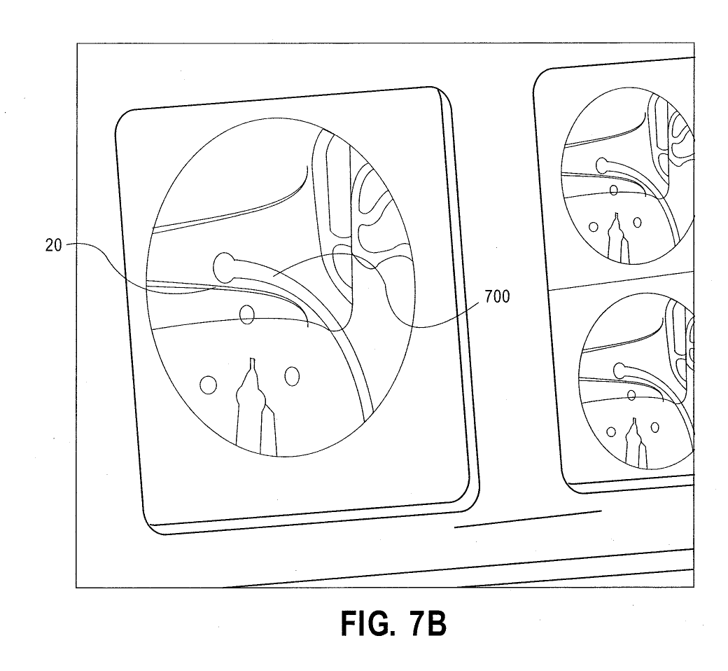

FIGS. 7a-7b are fluoroscans illustrating a device 700 with a reamer 710 advancing into the bone space of a patient; guides 20 are visible and also described in co-pending application 60/866,920 to Jobson for SURGICAL STATION FOR ORTHOPEDIC RECONSTRUCTIVE SURGERY. In the procedure shown, reamer 710 enters the distal end (distal relative to the patient) of the patient's radius bone at the lateral bony protuberance. A curved passage into the bone is first formed using an arc canula, as described above. The arc cannula is then held in a fixed position while reamer 710 is telescoped through it to form a generally straight path along a portion of the intramedullary canal of the bone. Telescoping is the action of sliding the flex reamer 500 in or out of arc canula 530.

In some procedures of the invention, a curved trocar forms the initial curved passage into the bone. A guidewire is then advanced through the curved passage into the intramedullary space, and its location is confirmed with fluoroscopy. Reamer 710 may then be advanced over the guidewire to enlarge the curved and straight passages in the bone. The use of the guidewire may be desirable when the passageways traverse multiple fracture lines which could cause an unguided reamer to deviate from its intended internal path and damage soft tissue outside the bone. After the passageways have been enlarged, the reamer and the guidewire may be removed together or individually. The guidewire may also be left in place after the reaming is completed and used to guide a bone splint or other devices into the bone passages. The guidewire may be provided with an enlarged distal (relative to the surgeon) end so that it may be used to withdraw a broken reamer. In some embodiments of the invention, two or more reamers of increasing diameter may be used in succession to create and/or enlarge a bone passage.

In use, the reamer creates bone chips as it creates or enlarges a passageway. According to aspects of the invention, it is desirable in many instances to leave the bone chips in the passageway that has been prepared for a bone splint. These bone chips contain hormones and bone growth factor that aid in the healing of bone fractures. Accordingly, the reamer head may be provided with a curved trailing surface (i.e. the leading surface closest to the arc canula when the reamer head is retreating). This curved shape, such as shown on the reamer head in FIGS. 1-4, tends to leave bone chips in place along passage walls rather than pushing them out with an auger effect or as a flat-faced reamer tends to do when being withdrawn. The ball shape of the reamer shown also contributes to more accurate cutting through the curved portion of the bone splint passage and keeps the cutting surfaces aimed towards the trajectory while minimizing unintended erosion of the bone surfaces on the sides.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A bone cutting instrument comprising:
a generally rigid arcuate tube having a generally fixed radius and a lumen therethrough;
a flexible drive shaft configured to be slidably and rotably received within the tube lumen; and
a cutter head attached to an end of the drive shaft, whereby the shaft and cutter head may be first advanced together with the tube in an arcuate manner to cut an arcuate path in a bone, and then advanced in a telescoping manner relative to the tube being held in a generally fixed position to cut a straight path in the bone.

2. The instrument of claim 1, further comprising a jig configured to be coupled with the arcuate tube for alternately advancing the tube in an arcuate manner and holding the tube in a generally fixed position.

3. The instrument of claim 2, wherein the jig is configured to be handheld.

4. The instrument of claim 2, wherein the jig is configured to be mounted in a fixed position relative to a surgical station.

5. The instrument of claim 1, wherein the cutter has a rounded end adjacent to the arcuate tube.

6. The instrument of claim 1, wherein the cutter has a non-flat shape on a end adjacent to the arcuate tube.

7. The instrument of claim 1, wherein the cutter has an approximately spherical shape.

8. The instrument of claim 1, wherein the cutter head and drive shaft include a continuous lumen therethrough.

9. The instrument of claim 8, further comprising a guide wire configured to be received through the lumen in the cutter head and drive shaft.

10. A method of forming a passage in a bone, the method comprising:
advancing a cutter head into a bone along a curved path having a generally constant radius; and
continuing to advance the cutter along a generally straight path extending from the curved path.

11. The method of claim 10, wherein the generally straight path is generally along a portion of an intramedullary canal of the bone.

12. The method of claim 10, wherein the curved path extends from an opening in a fractured bone into the bone.

13. The method of claim 12, where the opening is in a bony protuberance on an end of a radius bone.

14. The method of claim 10, further comprising advancing a curved trocar into the bone prior to advancing the cutter head into the bone.

15. The method of claim 14, further comprising advancing a guidewire along a path formed in the bone by the curved trocar and then using the guide wire to guide the cutter head along the curved path and the generally straight path.

16. The method of claim 10, further comprising creating bone chips inside the bone as the cutter is advanced.

17. The method of claim 16, further comprising removing the cutter head from the bone while leaving a majority of bone chips in the paths formed in the bone.

18. The method of claim 10, wherein the advancing of the cutter head along the curved path comprises pivoting the cutter head, a flexible drive shaft of the cutter head, and a curved tube receiving the drive shaft together about a common pivot point.

19. The method of claim 10, wherein the advancing of the cutter head along the generally straight path comprises holding a curved tube in a generally fixed position while extending a cutter head drive shaft from a lumen in the fixed tube.
